(19) United States
(12) Patent Application Publication
(10) Pub. No.: US 2013/0123145 A1
(43) Pub. Date: May 16, 2013

(54) METHODS. PARTICLES, AND ASSAY KITS FOR IDENTIFYING PRESENCE OF BIOLOGICAL PARAMETERS

(71) Applicants: Krishnan Chakravarthy, Williamsville, NY (US); Siddhartha Kamisetti, Cleveland, OH (US)

(72) Inventors: Krishnan Chakravarthy, Williamsville, NY (US); Siddhartha Kamisetti, Cleveland, OH (US)

(73) Assignee: NNANOAXIS, LLC, Clarence, NY (US)

(21) Appl. No.: 13/726,021

(22) Filed: Dec. 22, 2012

Related U.S. Application Data
(63) Continuation of application No. 13/517,577, filed on Jun. 13, 2012, now abandoned.
(60) Provisional application No. 61/496,202, filed on Jun. 13, 2011.

(51) Int. Cl. G01N 33/543

(52) U.S. Cl. CPC G01N 33/54346; Y10S 977/773; Y10S 977/92; Y10S 977/774; B82Y 10/00
USPC 506/16; 506/18; 435/7.92; 435/7.94; 436/501; 506/19; 435/7.24; 506/30; 977/773; 977/920; 977/774

(57) ABSTRACT

Multiplexed assays are disclosed for detection of duster differentiation 4 (CD4) glycoprotein expressed on the cell surface of I-helper cells, monocytes, macrophages, and dendritic cells; cluster differentiation 25 (CD25), a type transmembrane protein present on activated T-cells, activated B-cells, thymocytes, myeloid precursors, and oligodendrocytes; and Forkhead box P3 (FOXP3), an intracellular protein involved in immune system responses in cell cultures, tissues samples, humans, and biological samples. The multiplexed assays can be used to detect 1r-cells, activated I-cells, and other similar cell types (e.g. natural T regulatory cells, adaptive/induced T regulatory T cells). The multiplexed assays employ quantum dots of various shapes, types, compositions, coatings, sizes, ligands and other such characteristics.

### CROSS REFERENCED TO RELATED APPLICATION

This application is a continuation, that claims the priority benefit, of U.S. Non-Provisional application Ser. No. 13/517,577 filed Jun. 13, 2012, and entitled “Methods, Particles, and Assay Kits for Identifying Presence of Biological Parameters”, which claims the priority benefit of U.S. Provisional Application No. 61/496202, filed on Jun. 13, 2011, and entitled “Quantum Dot-Antibody Diagnostic Test for Protein Methylation for Rapid Clinical Cancer Detection and Prognosis”, the entirety of these applications are incorporated by reference herein in entirety.

### TECHNICAL FIELD

This disclosure relates to nanoparticle-based immunoassays for detecting biological substances such as proteins, DNA, RNA, PNA (peptide nucleic acid), microRNA, antigen, antibody, receptor, ligand, lectin, sugar-chain compound and other biological moieties.

### BACKGROUND

An assay kit can be utilized for a variety of purposes, such as, to count cells, measure cells, measure cell constituents, measure cell granularity, utilize fluorescence as a detection modality and conduct other research and development activities. Also, assay kits are used in the detection of various biological substances (e.g. cellular receptors) on a cell surface or intracellular substances (e.g. protein) through use of fluorescent labeling in order to assist a user in identifying a specific type of cell. All of these purposes are of great interest to researchers, developers, medical practitioners, and other assay kit users.

Various methods have been developed to identify antigen-specific T-cell responses. Traditional assays have analyzed bulk populations of T cells for proliferation or for cytotoxicity using fluorescent dyes. These assays tend to be long and labor-intensive, and the results usually cannot be compared quantitatively. Further, detection of cells with fluorescent labeling techniques often make use of organic dye molecules, attached to a biological substance, whereby the organic dye molecule luminesces when the dye is excited. The organic dye molecules provide inferior quantitative readouts due to factors including wide tail of emission spectrums, broad wavelengths, poor photostability and a limitation of colors leading to the detection of a few biological parameters at a single time.

### SUMMARY

The following presents a simplified summary of the disclosure in order to provide a basic understanding of sore aspects of the disclosure. This summary is not an extensive overview of the disclosure. It is intended to neither identify key or critical elements of the disclosure nor delineate any scope particular embodiments of the disclosure, or any scope of the claims. Its sole purpose is to present some concepts of the disclosure in a simplified form as a prelude to the more detailed description that is presented later.

In accordance with one or more embodiments and corresponding disclosure, various non-limiting aspects are described in connection with assay kits for the detection of biological parameters. In an aspect, the assay kit can detect the presence of various cell types including, but not limited to, T-helper cells, T-regulatory cells, TH-17 cells, and/or toll-like receptor expression.

In accordance with a non-limiting embodiment, in an aspect, an assay kit comprising: a set of hydrophilic coated nanoparticles paired to K biological materials that respectively form L nanoplexes that respectively bind to M predefined targeting moieties that correspond to N biological parameters and luminesce at respective wavelengths that correspond to the respective predefined biological parameter to determine P predefined target cell types, wherein K, L, M, N, and P are integers.

In an aspect, the hydrophobic coated nanoparticles are at least one of a: nanocrystal, quantum dot, spherical nanocrystal, non-spherical nanocrystal, spherical quantum dot, non-spherical quantum dot, tetrapod quantum dot, multi-leg luminescent nanomaterial, doped nanoparticle, polymer encapsulated quantum dot, or nanoparticle that exhibit luminescent properties. In another aspect, the quantum dot is at least one of a: heavy metal-free quantum dot, cadmium-free quantum dot, phosphorous quantum dot, or biocompatible quantum dot.

In yet another aspect, hydrophilic coat is any one or more of: Pluronic F127, silicon, micelle, glass, polymeric oxide, oxide of phosphorous, or polymeric ligands, amphiphilic ligand, or hydrophilic thiol compound. Furthermore, in an aspect, the predefined targeting moities are at least one of an: antibody, monoclonal antibody, polyclonal antibody, nucleic acid, monomeric nucleic acid, oligomeric nucleic acid, protein, polysaccharide, sugar, peptide, drug, RNA, DNA, microRNA, or plasmid. In another aspect, the hydrophilic coated nanoparticles are non-spherical quantum dots comprising a core material surrounded by a shell material that is aqueous solubilized and produced using a continuous flow chemistry process.

In an instance, the biological material is at least one of of: CD4 antibody, CD25 antibody, CD17 anitbody, TLR antibody, FoxP3 antibody, or methyl antibody. Furthmore, in an aspect, the predetermined target cell types are any one or more of: T regulatory cells, TH17 cells, T-cells or Toll-like receptors present on cells. Also, in an aspect the nanoplexes are packaged as a Z-plex assay kit, wherein Z is an integer. In another aspect, the assay kit is adapted for use in at least one of a lateral flow assay, ELISA, sandwich ELISA, microarray, piezoarray, Western blot, flow cytometry, or UV spectromphotometer.

The disclosure further discloses a method, comprising coating a set of nanoparticles to form a set of hydrophilic coated nanoparticles; pairing the V set of hydrophilic nanoparticles of one or more wavelengths to W biological materials to form X nanoplexes; V,W and X are integers.

### BRIEF DESCRIPTION OF THE DRAWINGS

FIG. 1 illustrates an example non-limiting assay kit for the detection of biological parameters,associated with predefined target cells.

FIG. 2 illustrates an example non-limiting multiplexing assay kit for the detection of biological parameters associated with dredefined target cells.

FIG. 3 illustrates an example non-limiting assay kit for the detection of biological parameters.

FIG. 4 illustrates an example non-limiting assay kit for the detection of biological parameters.

FIG. 5 illustrates an example non-limiting assay kit for the detection of biological parameters.

FIG. 6 illustrates an example methodology for coating, conjugating, binding to make an assay kit for the detection of biological parameters.

### DETAILED DESCRIPTION

### Overview

The innovation is now described with reference to the drawings, wherein like reference numerals are used to refer to like elements throughout. In the following description, for purposes of explanation, numerous specific details are set forth in order to provide a thorough understanding of this innovation. It may he evident, however, that the innovation can be practiced without these specific details. In other instances, well-known structures and components are shown in block diagram form in order to facilitate describing the innovation.

By way of introduction, the disclosed subject matter relates to assay kits for the identification of biological parameters (e.g. DNA, RNA, protein, antibody, etc.) and target cell types. To facilitate effective identification of biological parameters and cells, embodiments in this disclosure provide for one or more multiplexing assay kits to identify one or more biological parameters that correspond to the presence of one or more predefined target cell types in a biological sample. By identifying a cell type, a researcher, physician, medical practitioner, health care practitioner, patient, or other assay user are able to gather information and data regarding cell function (e.g. clearing of bacteria, secretion of hormone, propulsion, therapeutic effect, protection mechanism, sensory transduction, . . . ), cell therapy (effect on tissues and organs, repair of damaged muscle . . . ), tissue engineering, biomaterials engineering, growth factors, transplantation science, treatment of disease, mitigation of disease, treatment of injuries, immune system response, cell engineering (e.g. propagation of cells, expansion of cells, selection of cells, pharmacological treatment of cells, alteration of biological characteristics of cells . . . ), stem cells, embryonic cells, transplants, vaccines, drug delivery systems, cellular replication, controlling cellular mechanisms, isolating and purifying cell types, demonstrating clinical improvement, demonstrating cell development, and other important information and data relevant to conducting scientific research and development and promoting clinical pharmaceutical science.

Quantum dots are an advantageous nanoparticle for use in identifying biological paramaters in a biological substance. These nanoparticles present a variety of unique properties that provide utility in life science diagnostic applications, such as, controllable emission wavelengths, robust signal strength, sharp emission profiles, and excitation of multiple different quantum dots with a single excitation source. A quantum dot is characterized most simply by possessing a core comprising a first semiconductor material epitaxially surrounded by a shell comprising a second semiconductor material. The optical properties of quantum dots can be controlled by varying the core, size and composition of the quantum dot in order to produce quantum dots with a customizable range of emission spectra's (e.g. emission spectra's ranging from the ultraviolet spectra and spanning the near-infrared spectra).

Furthermore, the quantum dot shell and in some aspects, a coating, will affect core stability to produce a more stable photoluminescence result without affecting the emission range. The emission spectra produced by quantum dots are distinctive (e.g. possess a unique spectral signature) and emission peaks are much narrower than organic dyes thus presenting band width overlap (e.g. cross-talk) as opposed to traditional fluorophores such as organic dyes. Quantum dots can be produced in various shapes, sizes, and compositions, however, non-spherical quantum dots, particularly tetrapod shapes or multi-leg quantum dots offer superior properties to those of spherical quantum dots in relation to diagnostic assay kits.

One such advantage is the ability of multi-leg quantum dots to multiplex to a greater extent than spherical quantum dots. For example, unique spectral signatures of each multi-leg quantum dot are derived due to adjusting the number of legs presented on the nanoparticle, the width of the nanoparticle leg, or the length of the nanoparticle leg. Conversely, spherical quantum dots possess unique spectral signatures based only on adjusting the diameter of the quantum dot, thus presenting fewer customizable features that result in less varieties of unique spectral signature quantum dots.

The disclosed assay kit and several non-limiting embodiments make use of pairing hydrophilic nanoparticles (e.g. hydrophobic coated multi-leg luminescent nanoparticles) to biological material types to identify one or more predefined biological parameters assoicated with predefined target cell types. Thus the nanoparticle acts as a luminescent marker that tags various predefined biological materials. Furthermore, in an aspect, numerous multi-leg luminescent nanoparticles are used, each respectively emitting at a different wavelength (e.g. presenting different colors for imaging). Thus multiple biological parameters associated with a predefined target cell type may be tagged and can be used for phenotype identification of the target cell in addition to detecting levels of biological parameters present in a sample of interest.

For example, T regulatory cells are important for maintaining immune system tolerance to foreign antigens in a substance. T regulatory cells are associated with presence of extracellular proteins and intracellular proteins, whereby identification of these proteins simultaneously on and/or within a cell indicates the presence of a regulatory cell. The glycoprotein cluster of differentiation 4 (CD4) is expressed on the surface of helper cells, monocytes, macrophages, and dendritic cells. Cluster of differentiation 25 (CD25) is the alpha chain of the IL-2 receptor. It is a type I transmembrane protein present on activated T cells, activated B cells, some thymocytes, myeloid precursors, and oligodendrocytes.

Forkhead box P3 (Foxp3) is a protein involved in immune system responses. A member of the Fox protein family, Foxp3 appears to function as a master regulator in the development and function of regulatory T cells. Fox proteins belong to the forkhead/winged-helix family of transcriptional regulators and are presumed to exert control via similar DNA binding interactions during transcription. In human disease, alterations in numbers of regulatory T cells and in particular those that express Foxp3 are found in a number of disease states.

Together, the CD4, CD25, and Foxp3 antibodies can detect the presence of the CD4 glycoprotein, CD25 type I transmembrane protein, and intracellular Foxp3 protein in order to identify cell types such as regulatory T cells. Thus, in an aspect, three multi-leg nanoparticles, each emitting at a different wavelength (e.g. 530 nm, 560 nm, 590 nm) are respectively paired to a CD4 antibody, CD 25 antibody, and FoxP3 antibody. Each nanoparticle paired to a respective antibody (each individually referred to as a nanoplex) will target a respective CD4 receptor, CD 25 receptor, and FoxP3 intracellular protein associated with a I regulatory cell. Therefore, the presence of a 530 nm, 560 nm and 590 nm nanoparticle either on the surface or intracellular of a cell identifies the presence of a regulatory T cells present in a sample. To quantify the exact amount of regulatory T cells present in a sample of interest, the sample may be treated with a nanoplex comprising of hydrophilic nanoparticles paired to an antibody. The nanoplex may be excited using an ultraviolet energy source and detection of the nanoplex can be accomplished using a fluorometer. The concentration of regulatory T cells in a sample is determined based on a standard curve. The assay kit can be implemented in various assay formats for purposes of fluorescence detection including, but not limited to, flow cytometry, lateral glow, dual path platform, ELISA, sandwich ELISA, immunohistochemistry, FRET, or piezoarray for detecting nanoplex paired to biological materials in a sample post treatment

Identifying the presence of these three proteins can be valuable for research, development, diagnostic uses, and other applicable uses. In an aspect, the assay kits can be used to detect the movement of one or more biological substances from various intracellular or extracellular sites to an intracellular or extracellular destination. The assay kit can also be used to detect the presence of one or more biological substances in order to gather data and information relating to questions of importance in the fields of cell biology and medicine. Further, the assay kit can provide a simple method for a user to study mechanisms and biological factors that regulate cellular aspects including, but not limited to, metabolism or movement within a cell of a biological substance, intracellular signaling pathways pertinent to epigenetic analysis. Intracellular signaling pathways are particularly relevant with respect to detecting levels of methylation in a sample. Additionally, in several non-limiting embodiments, the assay kit can be presented in a variety of formats, including, but not limited to flow cytometry, immunocytochemistry, high content analysis, immunohistochemistry, fluorescent immunoassay, western blotting, and other such assays.

### Example Methods, Particles, and Assay Kits for Identifying Presence of Biological Parameters of Target Cells.

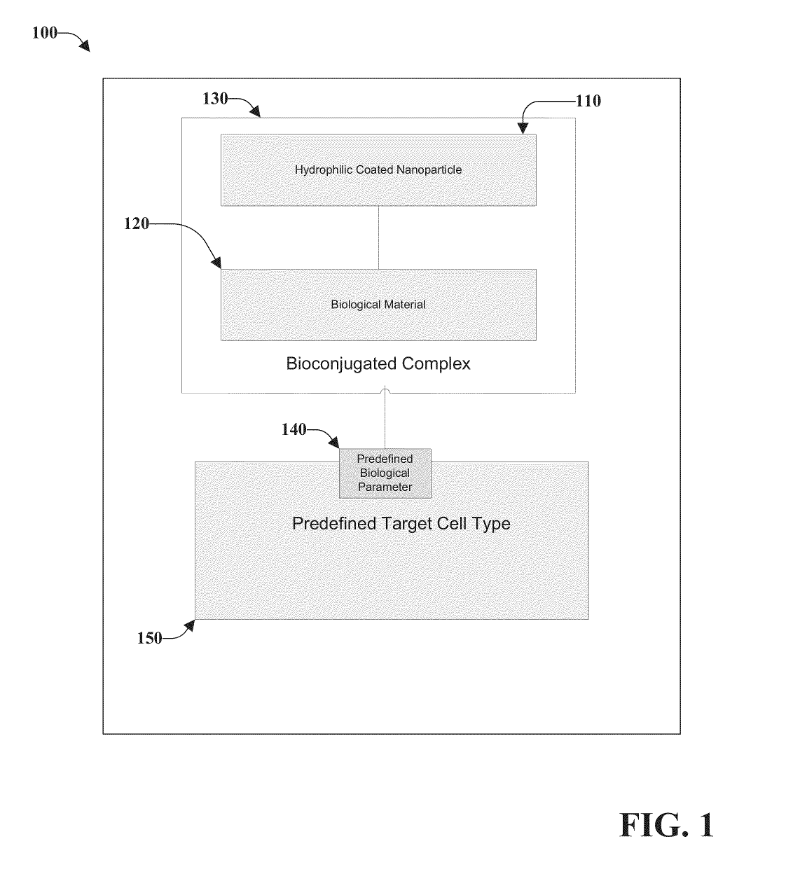

Referring now to the drawings, with reference initially to FIG. 1, assay kit 100 is shown that facilitates identifying predefined biological parameter of predefined target cell type based on a tagging with at least one nanoplex. Aspects of the systems, apparatuses, products, assay kits, processes or methods explained in this disclosure can constitute one or more embodiments with one or more components.

In an embodiment, assay kit 100 employs a hydrophilic coated nanoparticle 110, a biological material 120,a nanoplex 130, a predefined biological parameter 140, and a predefined target cell type 150. In an aspect, hydrophilic coated nanoparticle 110 is conjugated to biological material 120 to form nanoplex 130. The biological complex 130 binds to predefined biological parameter 140 and the nanoplex 130 tags predefined biological parameter 140 to identify predefined target cell type 150.

A nanoparticle is a particle ranging in size from 0.001 nm to 999.999 nm, of any shape, any size distribution, any form, and any material compositions. The size of a nanoparticle allows for selective advantages over other particles such as bulks materials. A variety of diagnostic advantages exist due to the size dependent properties of the nanoparticles and hydrophilic nanoparticles including, but not limited to, small sample amount requirements for analysis (e.g. greater sensitivity to enable detection of low-abundance targets), greater detection accuracy due to the availability of transmission light microscopy use, durable nanoparticle's that do not quench (e.g. quantum dots don't photo-bleach to the extent of organic dyes), the ability to use numerous colored nanoparticles to detect multiple biological targets simultaneously, narrow emission wavelengths in order to limit spectral overlap between adjacent colors. Additionally, nanoparticles offer selective optical, magnetic, and electrostatic advantages that larger particles don't possess. The hydrophilic coated nanoparticle 110 is a particle with dimensions ranging in size from 0.001 nm to 999.999 nm, wherein the nanoparticle can be suspended in aqueous solution.

Nanoparticles, such as multi-leg luminescent nanomaterials and quantum dots, must be made water soluble for use in biological applications. There are a number of methods for making a quantum dot or multi-leg luminescent nanomaterial hydrophilic. In an aspect, hydrophobic ligands on the surface of the multi-leg luminescent nanomaterial are substituted with hydrophilic ligands. In another aspect, hydrophobic surface ligands adsorb amphiphilic polymers containing both hydrophilic segments and hydrophobic segments (e.g. polyethylene glycol (PEG)). For example, in an aspect, a hydrophilic coated nanoparticle 110 can be a multi-leg luminescent nanomaterial with a carboxylated. PluronicF127 (F127COOH) triblock polymeric micelle (that for instance, surrounds, encapsulates, covers all or part of the nanoparticle surface).

In an aspect, the PluronicF127 hydrophilic coating can alleviate potential toxicity, enhance stability and improve targeting efficiency of the multi-leg luminescent nanomaterials. In another aspect, the hydrophilic coated nanoparticle 110 can comprise of any shape, size (within the 000.001 to 999.999 nm range), material composition, form, compound, or structure. For example, in an aspect, hydrophilic coated nanoparticle 110 is an iron oxide magnetic nanoparticle with a carboxylated PluronicF127 hydrophilic coating. In another instance, hydrophilic coated nanoparticle 110 is a tetrapod quantum dot nanoparticle paired to an iron oxide nanoparticle, wherein the tetrapod quantum dot-iron oxide nanoplex is coated with carboxylated Pluronic F127 coating.

In yet an aspect, hydrophilic coated nanoparticle 110 is a non-spherical or spherical quantum dot. The quantum dot can be comprised of a structured semiconductor core nanomaterial and structured semiconductor shell nanomaterial. The quantum dot is capable of emitting electromagnetic radiation and absorbing energy, and scattering or diffracting electromagnetic radiation when excited by an excitation source thus demonstrating a detectable and measurable change in absorption and emission radiation in a narrow wavelength band. Hydrophilic coated nanoparticle 110 can be comprised of one or more core and shell semiconductor nanoparticles, including, but not limited to; luminescent nanoparticle, multi-branched luminescent nanoparticle, snowflake shaped quantum dots, branched snow crystals, arrow shaped quantum dots, tetrapods, branched tetrapods, monopods, bipods, tripods, rods arrows, teardrops, disks, cubes, stars, pine-tree shaped, pyramids, pyramids, non-spherical, spherical, elipses, or other such quantum dot shapes or structures.

In an aspect, the hydrophilic coated nanoparticle 110 is a core and shell nanoparticle comprising a core material that is any one of a: Group 12, 13, 14, or 15 metal, Group II-VI semiconductor. Group II-V semiconductor, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe, GaAs, InGaAs, InP, InAs or metalloid; and comprising a shell material that is any one of (b) Group 12, 13, 14, or 15 metal, Group II-VI semiconductor, Group II-V semiconductor, MgS, MgSe, MgTe, CaS, CaSe, CaTe, SrS, SrSe, SrTe, BaS, BaSe, BaTe, ZnS, ZnSe, ZnTe, CdS, CdSe, CdTe, HgS, HgSe, HgTe, GaAs, InGaAs, InP, InAs or metalloid. The core/shell quantum dot has a hydrophilic coating surrounding the shell material in part or in full. In another aspect, hydrophilic coated nanoparticle 110 can be any one or more of a lipid, biodegradable polymer, gold nanoparticle, silver nanoparticle, iron oxide nanoparticle,

In an aspect, hydrophilic coated nanoparticle 110 comprises any one or more of a variety of biocompatible surface coatings such as ligand exchange, amphiphile encapsulation, and amphiphilic polymer coatings, as well as other coatings. Ligand exchange means in the case of a quantum dot, stripping away the quantum dot surface and replacing the surface with bifunctional capping molecules (e.g. 1-thioglycolic acid, 1-thioglycerol, mercaptoethylamine, L-cysteine, 3-mercaptopropoinic acid, N-acetyle-L-cysteine, dihydrolipoic acid, . . . ). Amphiphile encapsulation occurs where a nanoparticle is encapsulated in an amphiphile (e.g. DSPE phospholipids, 1,2-distearoyl-sn-glycero-3-phosphoethanolamine-N-[methoxy[poly(ethylene glycol)]], DSPEmPEG 5000, 1,2-distearoyl-sn-glycero-3-phosphoethanolamine-N-[amino(poly(ethylene glycol))2000], DSPE-PEG 2000 Amine, cyclodextrin). These coatings can transfer quantum dots into water.

In another aspect, hydrophilic coated nanoparticle 110 is a luminescent tetrapod dot is a nanomaterial compound comprised of (1) a structured semiconductor nanomaterial capable of emitting electromagnetic radiation and absorbing energy, and scattering or diffracting electromagnetic radiation when excited by an excitation source such as an electromagnetic radiation source or a particle beam which can demonstrate a detectable and measurable change in absorption and of emitting radiation in a narrow wavelength band or scattering of diffraction when excited. Only one common source for excitation of several tetrapod dots need be used due to the broad bandwidth of the nanomaterials, that is, several tetrapod dots give off radiation at different frequencies, thereby permitting simultaneous excitation.

In yet another embodiment, hydrophilic coated nanoparticle 110 is any one or more of a non-spherical quantum dot, including, but not limited to, arrow-shaped nanocrystal particles. It is understood that “arrow-shaped” nanocrystal particles can include tree-shaped nanocrystal particles such as pine-tree shaped nanocrystal particles. In other embodiments, non-spherical quantum dots, through various systematic manipulations, can take the shape of tetrapods, branched tetrapods, monopods, bipods, tripods, rods, arrows, teardrops, disks, cubes, stars, pine-tree shaped, pyramids, branched nanocrystal particles with a core and at least one arm extending from the core, pyramids, or any other suitable structure.

Luminescent tetrapod dots are capable of multiplexing based on the arm length and width. Multiplexing in this instance, is the simultaneous amplification, labeling and detection of many different targets with one single excitation source. In an aspect, the tetrapod quantum dots allow for the tracking of more than one biological parameters given the distinguishing characteristics afforded by these particles. Furthermore, the tetrapod quantum dot is capable of the arm length and width being adjustable as shorter, longer, thicker or thinner, which allows for additional emission wavelengths based on one source excitation with electromagnetic radiation or blue light. The enhanced optical properties are more robust when compared to standard spherical luminescent quantum dots with a smaller width at half maximum wavelength.

Furthermore, multiple analytes can be identified concurrently by use of many tetrapod dot nanomaterial compounds each tetrapod dot of which may emit at various wavelengths due to varying arm lengths, arm widths, apex diameters, material compositions, and productions of several tetrapods of such varying arm lengths, arm widths, apex diameters, all of which are excitable by one single excitation source. This offers a significant advantage in that each tetrapod quantum dot is capable of possessing a unique spectral signature by adjusting any of four features, arm length, arm width, number of arms, or apex diameter. Conversely a spherical quantum dot only possesses a unique spectral signature based on the adjusting of one feature, adjustment of the spherical diameter. This difference allows for greater multiplexing applications availble for tetrapod quantum dots due to more available tetrapod quantum dots with unique spectral signatures versus spherical quantum dots.

In one embodiment, the assay kit 100 uses the idea of attaching many colors of tetrapod quantum dots to different respective antibodies in order to obtain a distinctive combination of wavelengths. If five different antibodies for instance have five different wavelength respective tetrapod dots paired to its surface, each tetrapod dot emitting at a different wavelength, then an instrument can detect the nature of such target, by reading the five wavelengths emitted from such target in sequence. Likewise, sequence variations of tetrapod quantum dots with different wavelengths attached to targets can result in hundreds of paramaters being tagged in a single instance.

In another embodiment, each tetrapod can be produced in such a customized manner to possess different arm lengths, different arm widths, different core and shell materials, each arm and/or apex of which possess the ability to emit at different wavelengths all the while belonging to the same tetrapod quantum dot. Thus, in an aspect, hundreds of tetrapod quantum dots may be customized to track hundreds of targets in one single sample.

One of the selective optical properties of tetrapod dots that serve an important biological advantage is that the half maximum width of tetrapod dots are smaller than the conventional spherical quantum dots: the benefits of this is that it's easier to decipher more parameters when conducting real time analysis or multiplexing work due to more emission spectrums that can fit for a defined range of detection wavelengths.

In an aspect there are many applications that can be created by pairing tetrapod quantum dots to biological materials. For instance, by binding tetrapod quantum dots to both monoclonal and polyclonal antibodies we can create novel assay kits that will help create enhanced biological detection tools that work on the premise of tetrapod dots replacing existing biological dyes bound to antibodies. By using non-spherical quantum dot technology we achieve increased sensitivity, require less sample volumes, and measure multiple antigens in real time. Existing diagnostic techniques in the biological sciences are enhanced by these properties including but not limited to: flow cytometry applications, IHC, lateral flow assays, Western Blot, ELISA, microarray, PCR arrays, peizoarrays, and other such diagnostic tests.

In an aspect, hydrophilic coated nanoparticle 110 is a tetrapod dot. An advantage of tetrapod quantum dots as a diagnostic tool are its ability to change the composition of a single unit whereby the composition of the rods are made from different materials than the central sphere, and can continue to develop this for more complex inorganic dendrimers. In one aspect, the tetrapod quantum dot incorporates magnetic properties in the rods with the core being bioluminescent to allow for more elegant forms of cellular targeting. In a multi-chemistry paradigm used to provide selective targeting to a cell of interest both the rods and spheres may be independently manipulated even though they can be connected spatially.

In yet another aspect, hydrophilic coated nanoparticle 110 is a multi-leg luminescent nanoparticle. A multi-leg luminescent nanoparticle is a nanomaterial comprised of (1) a structured semiconductor nanomaterial capable of emitting electromagnetic radiation and absorbing energy, and scattering or diffracting electromagnetic radiation when excited by an excitation source such as an electromagnetic radiation source or a particle beam which can demonstrate a detectable and measurable change in absorption and of emitting radiation in a narrow wavelength band or scattering of diffraction when excited. Only one common source for excitation of several multi-leg luminescent nanoparticle need be used due to the broad bandwidth of the nanomaterials, that is, several multi-leg luminescent nanoparticle give off radiation at different frequencies, thereby permitting simultaneous excitation with a single common source.

A multi-leg luminescent nanoparticle comprises a base and one or more legs protruding from the base. Each multi-leg luminescent nanoparticle has ‘C’ number of legs extending from a base material, wherein “C” in an integer. The multi-leg luminescent nanoparticle has leg lengths which can be adjusted for each unique multi-leg luminescent nanoparticle, the leg length can range anywhere from 0.001 nm to 999.999 nm. The multi-leg luminescent nanoparticle has leg widths which can be adjusted for each unique multi-leg luminescent nanoparticle, the leg width can range anywhere from 0.001 nm to 999.999 nm. Furthermore, The multi-leg luminescent nanoparticle has base lengths which can be adjusted for each unique multi-leg luminescent nanoparticle, the base length can range anywhere from 0.001 nm to 999.999 nm.

Each nanoparticle may be constructed with legs of different leg lengths, legs of the same length or a mixture of same length, different leg length, same width, different width, same base length, and or different base length. Each combination of leg length, leg width, base length and or number of legs characterizing each multi-leg luminescent nanoparticle results in numerous different spectral signature outputs for each unique multi-legged luminescent nanoparticle. Each combination of varying leg lengths results in the ability to multiplex and account for multiple parameters at the same time. Additionally the leg width may be adjusted in distance to results in a different spectral signature output for each respective change in leg width. The nanoparticle also emits at different spectral signature outputs, which correspond to increasing or decreasing the non-leg lengths as well.

The ability to engineer each multi-leg luminescent nanoparticle with a one or more of legs of unique leg lengths, leg widths, and base lengths results in the ability to multiplex for thousands of parameters. Where each multi-leg luminescent nanoparticle is grown or assembled into multi-branched luminescent nanoparticles the multiplexing capability is increased exponentially.

In an embodiment, hydrophilic coated nanoparticle 110 is a multi-branched luminescent nanoparticle compound. A multi-branched luminescent nanoparticle compound is comprised of two or more multi-leg luminescent nanoparticles affixed through bonding, magnetics, and or complexation. A multi-branched luminescent nanoparticle can either be affixed to or protruding from another multi-branched luminescent nanoparticle. An attached multi-branched luminescent nanoparticle occurs where any leg of a multi-branched luminescent nanoparticle is attached to a base of another multi-leg luminescent nanoparticle through bonding, complexation or magnetics.

The multi-leg luminescent nanoparticle are special in that when they are prepared with a specific shell preparation and surface chemistry, they can be used in many applications, particularly for use in biological diagnostic assay kits. The optical properties, material composition, shape and structure of the compound, all allow for variance and flexibility in the properties of the multi-leg luminescent nanoparticle. The teachings of U.S. Provisional Pat. App. No. 61/538961 filed on Sep. 26, 2011 and U.S. Provisional Pat. App. No. 61/515468 filed on Aug. 5, 2011 are herein incorporated by reference.

In an aspect, the hydrophilic coated nanoparticles 110 may be a multiplexing assay formats can be produced using a continuous flow chemistry process. This process can produce nanoparticles such as non-spherical quantum dots (e.g. tetrapod quantum dots) or multi-leg luminescent nanomaterials in large scale (with core and/or core/shell systems and associated processed to make the respective nanoparticles hydrophilic. The process also includes a step to bind the nanoparticle to an antibody such that large-scale commercial production of assay kits are achieved. The current yield that can be achieved by producing the multi-leg quantum dot within the microreactor may be over 100 kg/reactor. However, parallel microreactors producing nanoparticles can increase the production quantities of the nanomaterials.

In an aspect, ligands are used as an adequate coating for hydrophilic coated nanoparticle 110. An amphiphile encapsulation provide for a suitable hydrophilic coating, however, a limitation with this coating is the instability of nanoparticles in biological environments because of relatively weak anchoring of the single and double hydrophilic tails to the particle. Amphiphilic polymers are chemical compounds possessing both hydrophilic and lipophilic properties. Amphophiles can potentially be used to tailor both hydrophilic and hydrophilic interactions between nanoparticles and its coating. As single polymer chains contain multiple hydrophilic units, the chains interact with the natural organic coatings on the surface of quantum dots, thus forming encapsulations that are often stronger than ligands or amphiphiles. Also, in yet another aspect, the external functionality of hydrophilic coated nanoparticle 110, can be functionalized through the introduction of molecules such as carboxyl or amino groups, on the hydrophilic moieties.

In an aspect, hydrophilic coated nanoparticle 110 can contain a hydrophilic coating comprising of Pluronic F127. In these F127 micelles, poly(propylene oxide (PPO) and ply(ethylene oxide)(PEO) serves as the hydrophilic section and poly(ethylene oxide(PEO) acts as the hydrophilic section. In another aspect, the hydrophilic coating can be glass or in part glass, such as silica, SiO, SiO2. Also, the hydrophilic coating can be in total or in part, polymeric oxide, oxide of silicon, oxide of boron, oxide of phosphorus, or a mixture of any one or more oxide. In yet another aspect, the hydrophilic coating can be in part or in whole, any one or mixture of metal silicate, metal borate, or metal phosphate. Other hydrophilic coatings include, but are not limited to, trioctylphosphine (TOPO), ethylene glycol, alkylithio acid, mercaptoacetic acid, or any combination of these coatings.

In another aspect, the hydrophilic coated nanoparticles 110 comprises functional groups on the nanoparticle surface (e.g. functional groups added to the shell or coating of the nanoparticle). Functional groups are chemistry groups positioned on the surface of hydrophilic coated nanoparticle 110. In an aspect, the hydrophilic coated nanoparticle 110 is any one or more of a quantum dot (e.g. tetrapod quantum dot, non-spherical quantum dot, spherical quantum dot etc.), muti-leg luminescent nanomaterial functionalized with any one or more chemical groups, or any combination of chemical groups, including, but not limited to, amino groups, carboxyl groups, azide groups, alkyne groups, hydrazine groups, aldehyde groups, aminooxy groups, ketone groups, maleimide groups, thiol groups, or other such chemical groups. In an aspect, hydrophilic coated nanoparticles 110 is paired to biological materials 120 to form nanoplex 130. In an aspect, a functional group is directly applied to a core of nanoparticles 110. In another aspect, a functional group is directly applied to a shell layer of nanoparticle 110. In another aspect, a functional group is directly applied to a coating of nanoparticle 110.

In an embodiment, biological material 120 is any material possessing a biological function. The biological material 120 can be any one or more endogenously-synthesized compounds that may influence biological phenomena or represent quantifiable biomarkers. In an aspect, biological material 120 can include, but is not limited to, any of a variety of biological substances such as antigens, biological markers, blood coagulation factor inhibitors, blood coagulation factors, chemotactic factors, inflammation mediators, intercellular signaling peptides, intracellular proteins, pheromones, pigments, biological toxins and other such biological substances. In another aspect, a biological material 120 can also be any one or more complex pharmaceutical substances, preparations, or agents of organic origin, usually obtained by biological methods or assay.

Furthermore, biological material 120 can be any one or more of an antibody, monoclonal antibody, nucleic acid, protein, polysaccharide, sugar, peptide, drug, oligomeric nucleic acid, or monomeric nuclic acid. The hydrophobic coated nanoparticles paired to a biological material 120 is known as a nanoplex. In an embodiment, a nanoplex is used in a biological or medical analysis system. In an aspect, a nanoplex is used to evaluate and diagnose disease states of tissue, either in vivo or in vitro. In another aspect, the nanoplex is used for medical imaging applications. In yet another aspect, the nanoplex is used for diagnostic assay kits. In an instance, the nanoplex contacts one or more predefined biological parameters. Upon contact, the nanoplex is exposed to a wavelength of light (e.g. ultraviolet light of a shorter wavelength than the respective emission wavelength of the nanoplex) that causes the nanoplex to luminesce and can he detected based on changes in fluorescence.

In an embodiment, the nanoplex 130 comprising of hydrophilic coated nanoparticle 110 paired to biological materials 120 can identify a predefined biological parameter 140. Nanoplex 130 can identify a predefined biological parameter 140 by tagging the predefined biological parameter. Tagging occurs when one or more nanoplex 130 is matched with one or more respective predefined biological parameter 140. A nanoplex 130 can pair with a predefined biological parameter 140 by various mechanisms including, but not limited to, magnetic attraction, attractive forces, bonding, mechanical bonding, electrostatic attraction, chemical bonds, covalent bonds, ionic bonds, hydrogen bonds, Van der Waals' forces, lock and key mechanism, and other such mechanisms.

The tagging of a biological parameter by a nanoplex can occur in many ways, one such way occurs through an antibody-receptor bonding. An antibody is a blood protein produced in response to and counteracting a specific antigen. Antigens are foreign molecular structures (e.g. a surface protein on a virus, a surface protein on a bacteria, an animal toxin, a surface protein on human tissue cells, etc.) and accordingly particular antibodies have an affinity for particular antigens and will bind to such antigen upon contact. An antibody can also associate with a receptor (located extracellularly or intracellularly) often times through an antibody-receptor bond. An antibody can perform various functions, all simply by binding to an antigen and/or a receptor. For instance, many viruses and some bacterial toxins affect cells by binding to receptors on cells. This either alters cell function (in the case of toxins) or allows entry into the cell (in the case of viruses). An antibody that binds to an antigen can block the virus or bacterial toxin and stop such virus or bacterial toxin from binding to a cell. For purposes of a diagnostic assay kit, an antibody that occupies a receptor site will luminesce and act as a tagging marker for such receptor site.

In an aspect, where the assay kit is a flow based assay, the antibody serves as biological molecule that binds to the desired biological parameter targeted for detection in the sample. Flow based detection happens whereby the number of events correlates with the number of cells paired to the nanoplex through the antibody. The nanoplex would be composed of an 1:1 ratio of antibody to quantum dots, therefore allowing for accurate measurement of concentration based on the number of events detected on a flow reading. In addition, if a predefined target cell is useful for phenotyping, which requires more than one biological parameter for detection; a cocktail of nanoplexes can be used whereby each wavelength of quantum dots is paired to a specific antibody marking a specific biological parameter on a predefined target cell. The combination of quantum dots paired to biological materials will allow an flow event to include all parameters at the same time with minimal compensation due to the quantum dots having narrow emission wavelengths.

In the case of devising an assay kit that detects protein methylation, the assay kit can work as fellows: the quantum dot paired to a methylation antibody may be bound at 1:1 stoichiometry ratio. The quantum dot paired to the antibody detects the presence of CH3 intra-celluarly and may be imaged using confocal microscopy. The intensity of the quantum dot emission signal correlates to the concentration of methylation within a predefined target cell. The antibody serves the role of a capture antibody in an ELISA or sandwich ELISA. This binding of the capture or detection antibody to the quantum dot allows it to used in various antibody driven assay formats that include Western Blots, immuno-histochemistry, lateral flow assays, flow based assays, ELISA, and sandwich ELISA, whereby the quantum dots serve as a superior alternative to biological dyes.

An antibody can also function by opsonization, wherein an antibody can bind to a foreign antigen and “flag” such antigen for the immune system to destroy and/or swallow (e.g. macrophages can recognize an antibody and engulf the foreign antigen or particle upon such recognition). An antibody can also function by attracting macrophages and neutrophils in a process called chemotaxis. Another function of antibodies is to participate in the process of rupturing membranes of foreign cells known as cell lysis. This nonexhaustive description of the functions of antibodies provide insight into the useful nature of antibodies and contribute to an understanding of antibody-antigen or antibody-receptor bonding. In an aspect, nanoplex 130 comprising of a hydrophilic coated nanoparticle 110 paired to an antibody matches to a receptor specific to the antibody for purposes of tagging a predefined target cell type 150.

For instance, a nanoplex 130 can bind to a predefined biological parameter 140. One such example is a nanoplex comprising a hydrophilic tetrapod quantum dot conjugated to a CD4 antibody. The nanoplex can then match to a predefined biological parameter 140 comprising a CD4 antigen on a cell surface. The matching of the nanoplex to a CD4 antigen can occur through antibody-antigen bonding in this instance. In an aspect, upon bonding, the nanoplex bound to a biological parameter is imaged (e.g. through chemical photography, a UV spectrophotometer, a flow cytometer, transmission electron microscopy, confocal microscopy)

In another embodiment, the target cell type 150 is any type of cell associated with one or more biological parameter 140. Cells are the fundamental structural, and functional units or subunits of living organisms. Researchers (e.g. cell biology researchers, cancer researchers, etc.) are interested in studying cells for physiological properties, structure, cellular organelles, function, interaction with environment, life cycle, division, death, differentiating between cell types, and other such interests. In an aspect, assay kit 100 provides an efficient and effective assay to identify biological parameters associated with predefined target cell type 150. In an aspect, predefined target cell type 150 can include, but is not limited to, transformed cell lines, hybrid cell lines, tumor cell lines, stem cells, and other such cells. In another aspect, target cell type 150 can include any one or more of the cell types in the human body and animal bodies (e.g pig, rat, sheep, monkey, . . . ).

Further, predefined target cell types 150 can be any one or more gland cells such as exocrine secretory epithelial cells (e.g. salivary gland serous cell, sebaceous gland cell, etc.), hormone secreting cells (e.g. anterior pituitary cells, intermediate pituitary cell, magnocellular neurosecretory cells, gut and respiratory tract cells, throid gland cells, parathyroid gland cells, parathyroid gland cells, adrenal gland cells, leydig cell of testes, theca interna cell of ovarian follicle, circus luteum cell, etc.), epithelial cells lining dosed internal body cavities, ciliated cells with propulsive function, integumentary system cells (e.g. keratinizing epithelial cells, wet stratified barrier epithelial cells), nervous system cells (e.g. sensory transducer cells, autonomic neuron cells, sense organ and peripheral neuron supporting cells, central nervous system neurons and glial cells, lens cells, etc.), cells derived primarily from mesoderm metabolism and storage cells, barrier function cells (lung, gut, exocrine glands, urogenital tract), kidney cells, extracellular matrix cells, contractile cells, blood and immune system cells, pigment cells, germ cells, nurse cells, interstitial cells), and other such cell types.

Furthermore, target cell 150 can include any one or more of the following specific cell types including but not limited to, salivary gland mucous cell, salivary gland serous cell, Von Ebner's gland cell, mammary gland cell, lacrimal gland ceruminous gland cell in ear, eccrine sweat gland dark cell, eccrine sweat gland clear cell, apocrine sweat gland cell, Gland of Moll cell, sebaceous gland cell, Bowman's gland cell, Brunner's gland cell, seminal vesicle cell, prostate gland cell, Bulbourethral gland cell (mucus secretion), Bartholin's gland cell, Gland of Littre Uterus endometrium cell, Isolated goblet cell, stomach lining mucous cell, gastric gland zymogenic cell, gastric gland oxyntic cell, pancreatic acinar cell, Paneth cell, Type II pneumocyte, Clara cell, anterior pituitary cells, Somatotropes, Lactotropes, Thyrotropes, Gonadotropes, Corticotropes, Intermediate pituitary cell, Magnocellular neurosecretory cells, (e.g. secreting oxytocin, secreting vasopressin), Gut and respiratory tract cells (e.g. secreting serotonin, secreting endorphin, secreting somatostatin, secreting gastrin, secreting secretin, secreting cholecystokinin, secreting insulin, secreting glucagon, secreting bombesin)

Additionally, target cell 150 can be include, but is not limited to cell types such as Thyroid gland cells, thyroid epithelial cell, parafollicular cell, Parathyroid gland cells, Parathyroid chief cell, Oxyphil cell, Adrenal gland cells, chromaffin cells, secreting steroid hormones (mineralcorticoids and gluco corticoids), Leydig cell of testes secreting testosterone, Theca inferna cell of n follicle secreting estrogen, Corpus luteum cell of ruptured ovarian follicle secreting progesterone, Granulosa lutein cells, Theca lutein cells, Juxtaglomerular cell, Mactila densa cell of kidney, Peripolar cell of kidney, Mesangial cell of kidney, Blood vessel and lymphatic vascular endothelial continuous cell, Blood vessel and lymphatic vascular endothelial splenic cell, Synovial cell, Serosal cell (lining peritoneal, pleural, and pericardial cavities), Squamous cell, endolymphatic, Columnar cell of endolymphatic sac with microvilli, Columnar cell of endolymphatic sac without microvilli, Dark cell, Vestibular membrane cell, Stria vascularis basal cell, Stria vascularis marginal cell, Cell of Claudius, Cell of Boettcher, Choroid plexus cell, (Pia-arachnoid squamous cell, Pigmented ciliary epithelium cell, Nonpigmented ciliary epithelium cell, Corneal endothelial cell, Peg cell, Respiratory tract ciliated cell, Oviduct ciliated cell, Uterine endometrial ciliated cell, Rete testis ciliated cell, Ductulus efferens ciliated cell, Ciliated ependymal cell of central nervous system (lining brain cavities).

Furthermore, predefined target cell types 150 can include any one or more of the following specific cell types including but not limited to Epidermal keratinocyte (differentiating epidermal cell), Epidermal basal cell (stem cell), Keratinocyte of fingernails and toenails, Nail bed basal cell (stern cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Surface epithelial cell, basal cell (stern cell) Urinary epithelium cell (lining urinary bladder and urinary ducts) Auditory inner hair cell of organ of Corti, Auditory outer hair cell of organ of Corti, Basal cell of olfactory epithelium (stem cell for olfactory neurons), Cold-sensitive primary sensory neurons, Heat-sensitive primary sensory neurons, Merkel cell of epidermis (touch sensor), Olfactory receptor neuron, Pain-sensitive primary sensory neurons (various types), Photoreceptor cells, Photoreceptor rod cells, Photoreceptor blue-sensitive cone cell of eye, Photoreceptor green-sensitive cone cell of eye, Photoreceptor red-sensitive cone cell of eye, Proprioceptive primary sensory neurons (various types), Touch-sensitive primary sensory neurons (various types),

Additionally, target cell 150 can be include, but is not limited to cell types such as Type I carotid body cell (blood pH sensor), Type II carotid body cell (blood pH sensor), Type I hair cell of vestibular apparatus of ear (acceleration and gravity), Type II hair cell of vestibular apparatus of ear (acceleration and gravity), Type I taste bud cell. Cholinergic neural cell (various types), Adrenergic neural cell (various types), Peptidergic neural cell (various types), Inner pillar cell of organ of Corti, Outer pillar cell of organ of Corti, Inner phalangeal cell of organ of Corti, Outer phalangeal cell of organ of Corti, Border cell of organ of Corti, Hensen cell of organ of Corti, Vestibular apparatus supporting cell, Taste bud supporting cell, Olfactory epithelium supporting cell, Schwann cell, Satellite cell (encapsulating peripheral nerve cell bodies),

Additionally, predefined target cell types 150 can be include, but is not limited to cell types such as Enteric glial cell, Astrocyte (various types), Neuron cells (large variety of types, still poorly classified), Oligodendrocyte, Spindle neuron, Anterior lens epithelial cell, Crystallin-containing lens fiber cell, Hepatocyte (liver cell), Adipocytes, White fat cell, Brown fat cell, Liver lipocyte, Kidney glomerulus parietal cell, Kidney glomerulus podocyte, Kidney proximal tubule brush border cell, Loop of Henle thin segment cell, Kidney distal tubule cell, Kidney collecting duct cell, Type I pneumocyte (lining air space of lung cell), Pancreatic duct cell (centroacinar cell), Nonstriated duct cell (of sweat gland, salivary gland, mammary gland, etc.), principal cell, Intercalated cell, Duct cell (of seminal vesicle, prostate gland, etc.), Intestinal brush border cell (with microvilli), Exocrine gland striated duct cell, Gall bladder epithelial cell, Ductulus efferens nonciliated cell, Epididymal principal cell, Epididymal basal cell, Ameloblast epithelial cell (tooth enamel secretion).

Additionally, target cell 150 can be include, but is not limited to cell types such as Planum semilunatum epithelial cell of vestibular apparatus of ear (proteoglycan secretion), Organ of Corti interdental epithelial cell (secreting tectorial membrane covering hair cells), Loose connective tissue fibroblasts, Corneal fibroblasts (corneal keratocytes), Tendon fibroblasts, Bone marrow reticular tissue fibroblasts, Other nonepithelial fibroblasts, Pericyte, Nucleus pulposus cell of intervertebral disc, Cementoblast/cementocyte (tooth root bonelike cementum secretion), Odontoblast/odontocyte (tooth dentin secretion), Hyaline cartilage chondrocyte, Fibrocartilage chondrocyte, Elastic cartilage chondrocyte, Osteoblast/osteocyte, Osteoprogenitor cell (stem cell of osteoblasts)Halocyte of vitreous body of eye, Stellate cell of perilymphatic space of ear, Hepatic stellate cell (Ito cell), Pancreatic stelle cell, Skeletal muscle cells, Red skeletal muscle cell (slow). White skeletal muscle cell (fast), Intermediate skeletal muscle cell, nuclear bag cell of muscle spindle, nuclear chain cell of muscle spindle, Satellite cell (stem cell).

Furthermore, target cell 150 can include any one or more of the following specific cell types including but not limited to heart muscle cells. Ordinary heart muscle cell, Nodal heart muscle cell, Purkinje fiber cell, Smooth muscle cell (various types), Myoepithelial cell of iris, Myoepithelial cell of exocrine glands, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper cell, Suppressor T cell, Cytotoxic T cell, Natural Killer cell, B cell, Natural killer cell Reticulocyte, Stem cells and committed progenitors for the blood and immune system (various types), Melanocyte, Retinal pigmented epithelial cell, Oogonium/Oocyte, Spermatid, Spermatocyte, Spermatogonium cell (stem cell for spermatocyte), Spermatozoon, Ovarian follicle cell, Sertoli cell (in testis), Thymus epithelial cell, Interstitial kidney cells, and other such cells.

In an aspect, predefined target cell types 150 are identified by tagging with nanoplex 130, the biological parameter 140 associated with predefined target cell types 150. In an aspect, nanoplex 130 adheres to biological parameter 140, subsequently nanoplex 130 is excited by an energy source, whereby nanoplex 130 emits light at a specific wavelength. A diagnostic analysis of the wavelength emission of one or more nanoplex 130 indicates the presence of the predefined target cell types 150 associated with biological parameters 140. Thus the biological parameter 140 tagged with a nanoplex 130 that emits light at the wavelength of a respective nanoplex will identify the predefined target cell types 150 associated with the biological parameter 140. Furthermore, by noting areas of luminescence and differences in characteristics (e.g. luminescence intensity) resulting from different nanoplexes in different region of sample, the differences in characteristics (e.g. such as luminescence intensity) can be used to identify cell types, disease states in tissue (e.g. cancerous tissue) or other identification features.

For instance, protein arginine methylation is a rapidly growing field of biomedical research that holds great promise for extending our understanding of developmental and pathological processes. Type I and type II protein arginine methyltransferases (PRMTs) are known to be responsible for catalyzing the arginine methylation of human proteins. Less than ten years ago, fewer than two dozen proteins were verified to contain methylarginine. Currently, however, hundreds of methylarginine proteins have been detected. Several of these proteins are products of disease genes or are implicated in disease processes by recent experimental or clinical observations, such as neurodevelopmental diseases, autoimmune disorders, and viral, neoplastic and cardiovascular diseases.

In an instance, nanoparticle 110 is tetrapod quantum dot which possesses enhanced brightness and high photostability for high sensitivity diagnosis of multiple methylated proteins, using an advanced microarray based ELISA assay (Microarray Immunoassay, or MI). The traditional MI technique uses capture antibodies (e.g. methylarginine-specific antibodies), attached to nitrocellulose pads coated on glass slides, to capture corresponding antigens (analyte) in a fluid sample. In an aspect, biological materials 110 is an antibody that corresponds to methylated protein antigens. In an aspect, the tetrapod quantum dot-methyl antibody nanoplex 130 has an affinity for methyl antigens and binds to such methyl antigens. Thus methyl antigens associated with specific predefined target cell types 150 are labeled with corresponding tetrapod quantum dot-methyl antibody nanoplex 130 recognizing a different epitope. The tetrapod quantum dot-methyl antibodies nanoplex 130 are fluorescently labeled via a steptavidin-fluorophore linkage. The intensity of the fluorescence associated with the detection antibody quantifies the amount of the analyte. In an aspect, the emission intensities of the tetrapod quantum dot-methyl antibody-methylated antigen complex are used to quantify the corresponding analytes. In other instances, many nanoplexes of different wavelengths may be used to tag biological parameters and identify a predefined targe cell type.

In an aspect, assay kit 100 identifies predefined target cell type 150 by tagging more than one predefined target parameter 140. Multiplexing is the tagging of more than one unique biological parameters with a different light emitting nanoplex 130 for each respective unique biological parameter. For instance, a CD4 biological parameter can be tagged with a biological complex that emits light at a 530 nm wavelength, a CD25 biological parameter can be tagged with a biological complex that emits light at a 630 nm wavelength, and an intracellular Foxp3 biological parameter can be tagged with a biological complex that emits light at a 730 nm wavelength. Furthermore, a cell can be tagged with B number of biological parameters where B is an integer.

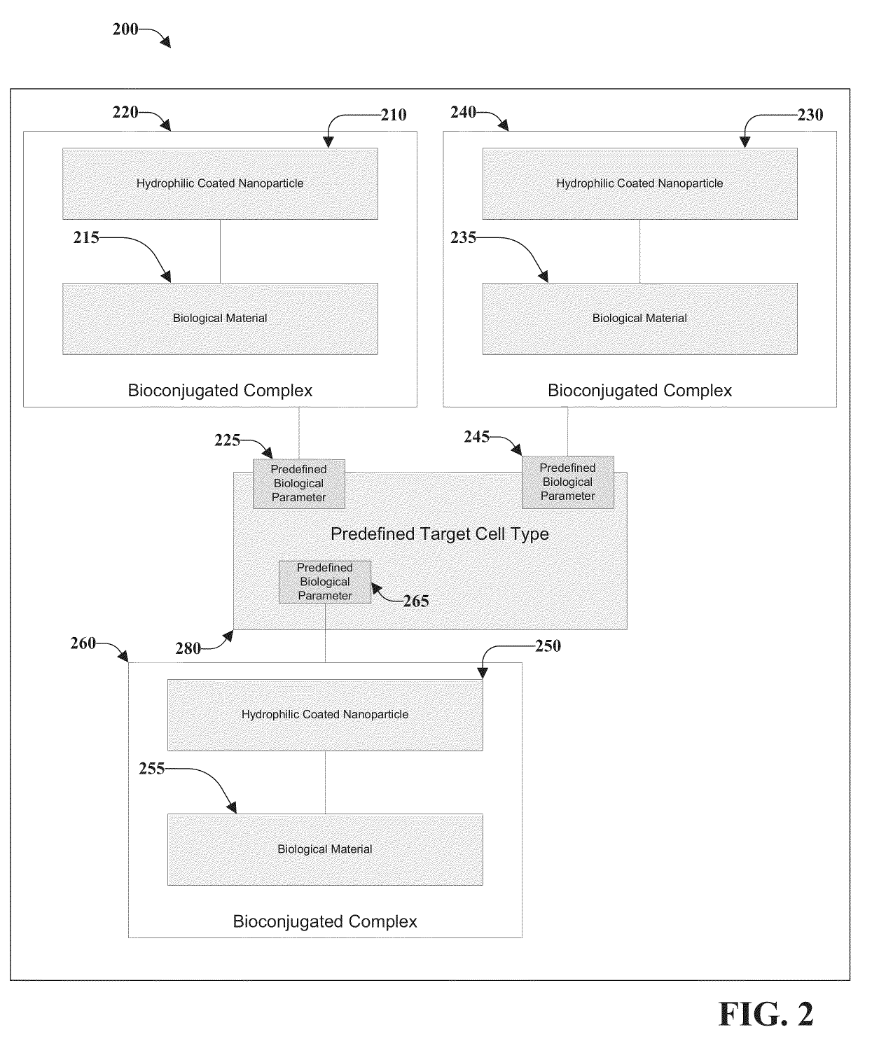

Turning now to FIG. 2, presented is another exemplary non-limiting embodiment of assay kit 200 in a predefined target cell type 280 by tagging one or more predefined biological parameters and identifying a predefined target cell type 280 by multiplexing. For instance, regulatory T cells are a component of the immune system that suppress immune responses to other cells. Regulatory T cells come in many forms but are commonly understood as cells that express CD4, CD25 and Foxp3 proteins.

A user could use an assay comprised of three nanoplex emitting light at three wavelengths comprising; a first nanoplex 220 comprising of a hydrophilic coated nanomaterial 210 paired to a biological material 215 wherein the hydrophilic coated nanomaterial 210 is a 530 nm tetrapod quantum dot and the biological material 215 is a CD4 antibody and the 530 nm tetrapod quantum dot is bioconjugated to the CD4 antibody, a second nanoplex 240 comprising a hydrophilic coated nanomaterial 230 paired to a biological material 2350 wherein the hydrophilic coated nanomaterial 230 is a 630 nm tetrapod quantum dot and the biological material 235 is a CD25 antibody and the 630 nm tetrapod quantum dot is bioconjugated to the CD25 antibody, a third nanoplex 260 comprising a hydrophilic coated nanomaterial 250 bioconjugated to a biological material 255 wherein the hydrophilic coated nanomaterial 250 is a 730 nm tetrapod quantum dot and the biological material 255 is a Foxp3 antibody and the 730 nm tetrapod quantum dot is bioconjugated to the CD25 antibody.

Further, each nanoplex corresponds to a specific predetermined biological parameter associated with a predefined target cell type. In the example, nanoplex 220 corresponds to predefined biological parameter 225, which, for the example, is a CD4 extracellular receptor. Furthermore, nanoplex 240 is associated with predefined biological parameter 245, which for our example is a CD25 extracellular receptor. Nanoplex 260 is associated with predefined biological parameter 265, which for our example is a Foxp3 intracellular receptor transcription factor. Each nanoplex can tag its respective corresponding biological parameter, in the example, nanoplex 220 tags predefined biological parameter 225 by attaching the CD4 antibody to the CD4 extracellular receptor.

Accordingly, in the example, nanoplex 240 tags predefined biological parameter 245 by attaching the CD25 antibody to the CD25 extracellular receptor. Also, in the example, nanoplex 260 tags predefined biological parameter 265 by attaching the Foxp3 antibody to the Foxp3 intracellular factor. A predefined target cell type 280 is identified when all three biological parameters are tagged. Thus, for the example, predefined cell type 280 can be a regulatory cell and the regulatory cell is identified when the cell is tagged with a 530 nm tetrapod quantum dot paired to a CD4 antibody, a 630 nm tetrapod quantum do paired to a CD4 antibody, and a 730 nm tetrapod quantum dot paired to a Foxp3 antibody simultaneously. Upon excitation of the three nanoplexes, the three wavelengths will be apparent through a wavelength reading from a UV spectrophotometer, a flowcytometer wavelength readier, an imaging technique through microscopic analysis, or through other such detection mechanisms that measure change in fluorescence.

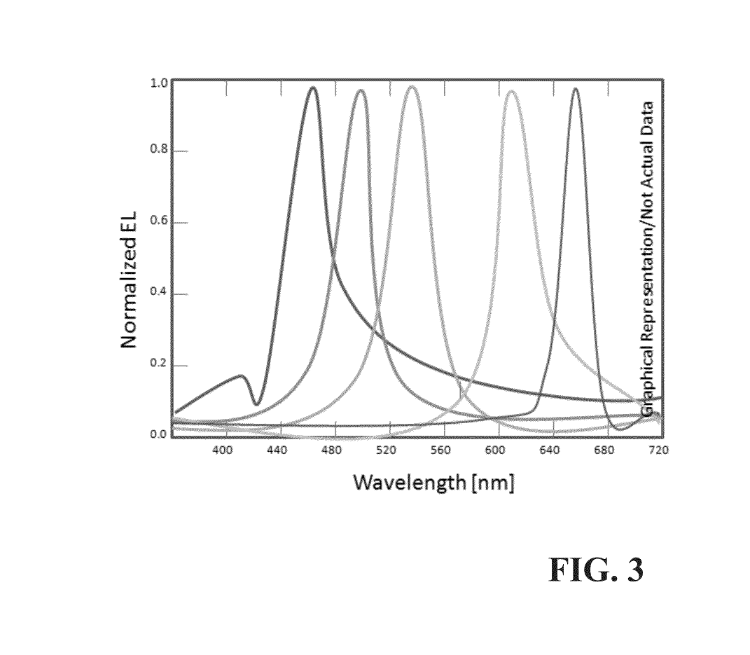

Now turning to FIG. 3, illustrated is a chart to illustrate how different nanoplex wavelengths are displayed in graphical form. Each wavelength corresponds to a nanoplex that emits at a different wavelength. The peaks are narrow so each nanoparticle (e.g. non-spherical quantum dot) of a different wavelength paired to a different respective biological parameter can be differentiated on a read out. The narrow peaks make the different nanoplexes differentiable. On the x-axis, the wavelength is plotted.

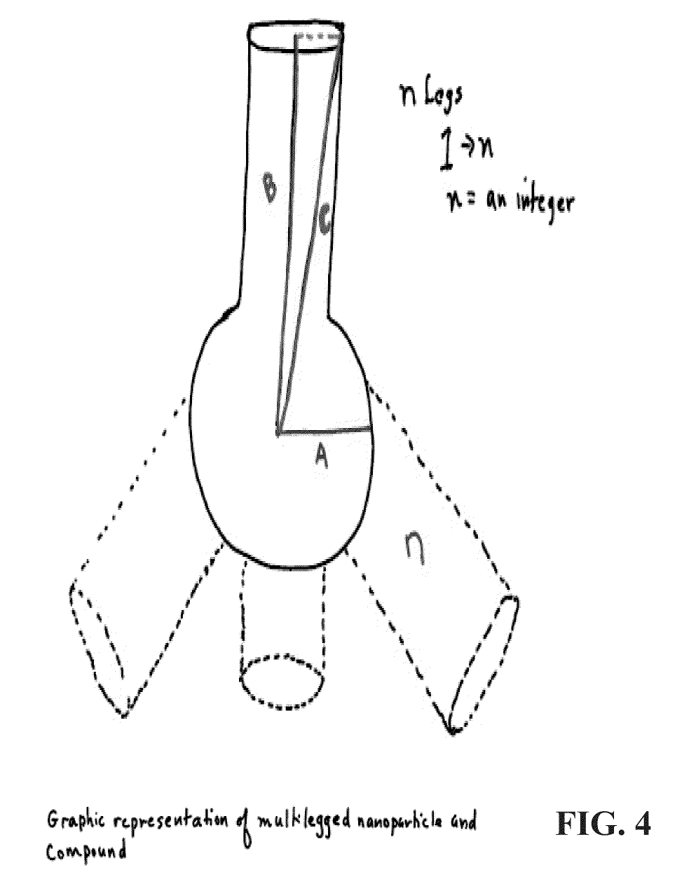

Now turning to FIG. 4, illustrated is a multi-leg luminescent nanomaterial. The multi-leg nanomaterial possesses N legs, wherein N is an integer. A multi-leg luminescent nanoparticle comprises a base and one or more legs protruding from the base. Each multi-leg luminescent nanoparticle has ‘n’ number of legs extending from a base material where “n” in an integer. The multi-leg luminescent nanoparticle has leg lengths (indicated by “B” in FIG. 4), which can be adjusted for each unique multi-leg luminescent nanoparticle, the leg length can range anywhere from 0.001 nm to 999.999 nm. The multi-leg luminescent nanoparticle has leg widths (indicated by “C” in FIG. 4) and FIG. 3) which can be adjusted for each unique multi-leg luminescent nanoparticle, the leg width can range anywhere from 0.001 nm to 999.999 nm. Furthermore, The multi-leg luminescent nanoparticle has base lengths (indicated by “A” in FIG. 4) which can be adjusted for each unique multi-leg luminescent nanoparticle, the base length can range anywhere from 0.001 nm to 999.999 nm.

Each nanoparticle may be constructed with legs of different leg lengths, legs of the same length or a mixture of same length, different leg length, same width, different width, same base length, and or different base length. Each combination of leg length, leg width, base length and or number of legs characterizing each multi-leg luminescent nanoparticle results in numerous different spectral signature outputs for each unique multi-legged luminescent nanoparticle. Each combination of varying leg lengths results in the ability to multiplex and account for multiple parameters at the same time. Additionally the leg width may be adjusted in distance to results in a different spectral signature output for each respective change in leg width. The nanoparticle also emits at different spectral signature outputs which correspond to increasing or decreasing the non-leg lengths as well.

The ability to engineer each multi-leg luminescent nanoparticle with a one or more of legs of unique leg lengths, leg widths, and base lengths results in the ability to multiplex for thousands of parameters. Where each multi-leg luminescent nanoparticle is grown or assembled into multi-branched luminescent nanoparticles the multiplexing capability is increased exponentially. Each multi-leg luminescent nanoparticle can have a different unique spectral signature and therefore can track for a different biological parameter when paired to an associated biological material.

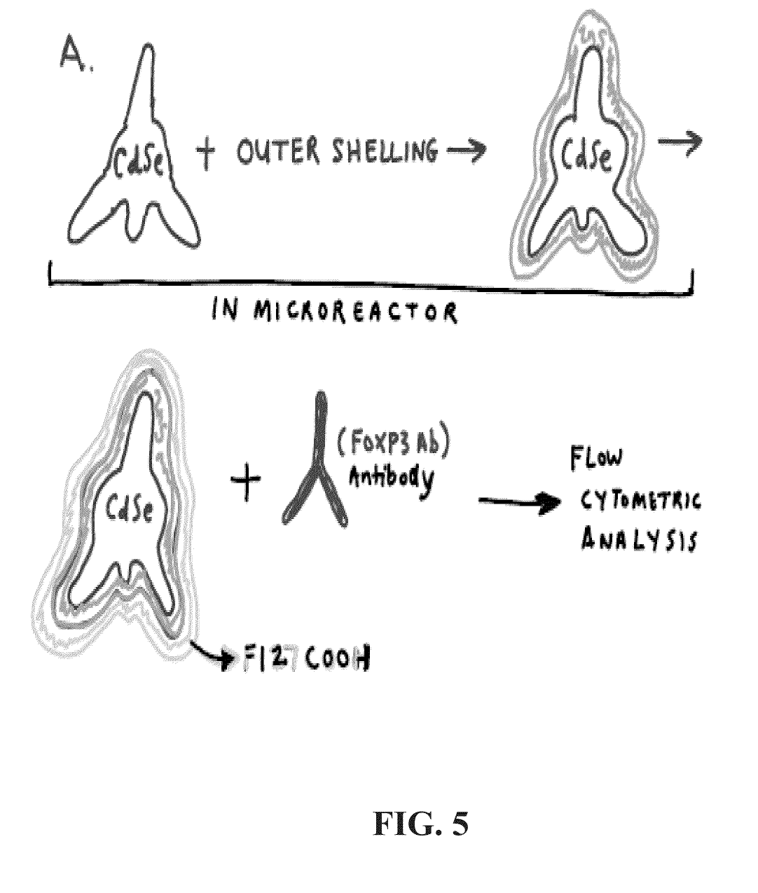

FIG. 5 illustrates the process of making a mult-leg luminescent nanoparticle biologically capable. An outer shell is added to the core, such shell can be added in a micororeactor flow chemistry process. Additionally a coating can be added to the shell. In another aspect, an antibody can be paired to the surface of the shell. All such steps can occur in a flow chemistry microreactor. The multi-leg luminescent nanoparticle can he analyzed in a flow cytometer.

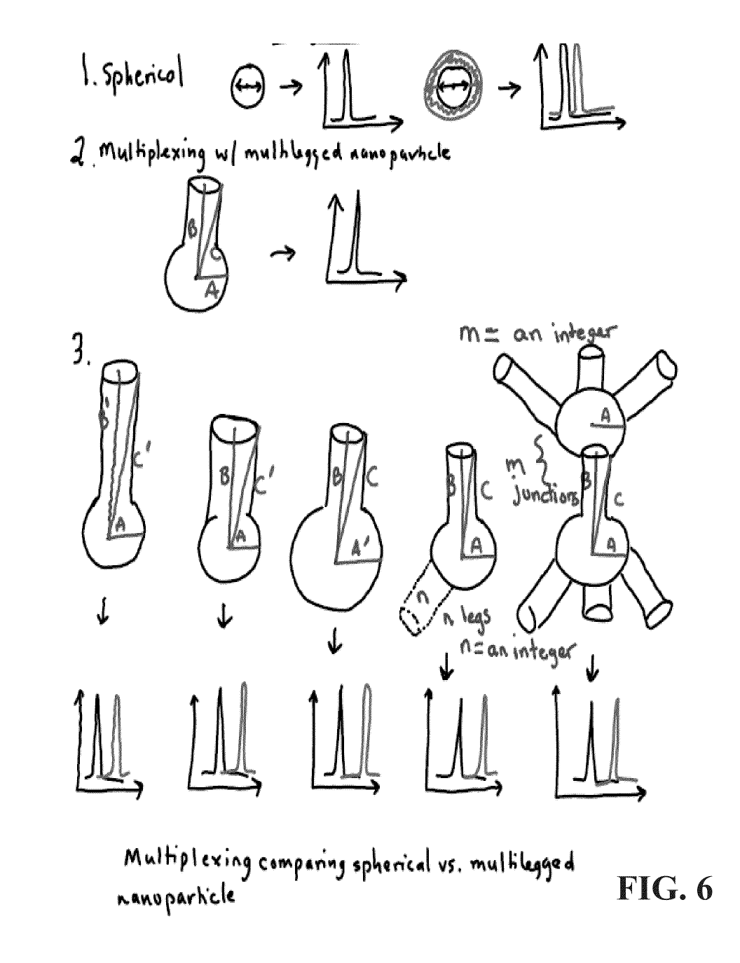

FIG. 6 illustrates the multiplexing by using a multi-leg luminescent nanomaterial versus a spherical quantum dot. More unique spectral signatures may be made by using a multi-leg luminescent nanomaterial rather than a spherical quantum dot. Additionally more parameters can be detected using the multi-leg luminescent nanomaterial. Furthermore, a greater number of multiplexing options are available with the multi-leg luminescent nanomaterial.

In accordance with the present invention, there are provided a method to produce nanoparticles, such as multi-leg luminescent nanoparticles in large quantities (in one aspect kilogram amounts). The nanoparticle can be produced through a micro-reactor process that utilizes continuous flow chemistry processes to produce nanomaterials of uniform size, shape, and composition distributions.

In summary, disclosed are methods, particles, and assay kits for identifying the presence of biological parameters. The embodiments are non-limiting and nothing in this specification is intended to limit the scope of the present invention. The described emodiments may be modified or varied, without departing from the invention. All examples presented are represtnative an are non-limiting.

1. An assay kit comprising:
a set of hydrophilic coated nanoparticles paired to K biological materials that respectively form L nanoplexes that respectively bind to M predefined targeting moieties that correspond to N biological parameters and luminesce at respective wavelengths that correspond to the respective predefined biological parameter to determine P predefined target cell types, wherein K, L, M, N, and P are integers.

2. The assay kit of claim 1, wherein the hydrophobic coated nanoparticles are at least one of a: nanocrystal, quantum dot, spherical nanocrystal, non-spherical nanocrystal, spherical quantum dot, non-spherical quantum dot, tetrapod quantum dot, multi-leg luminescent nanomaterial, doped nanoparticle, polymer encapsulated quantum dot, or nanoparticle that exhibit luminescent properties.

3. The assay kit of claim 1, wherein the quantum dot is at least one of a:
heavy metal-free quantum dot, cadmium-free quantum dot, phosphorous quantum dot, or biocompatible quantum dot.

4. The assay kit of claim 1, wherein the hydrophilic coat is any one or more of: Pluronic F127, silicon, micelle, glass, polymeric oxide, oxide of phosphorous, or polymeric ligands, amphiphilic ligand, or hydrophilic thiol compound.

5. The assay kit of claim 1 wherein the predefined targeting moities are at least one of an: antibody, monoclonal antibody, polyclonal antibody, nucleic acid, monomeric nucleic acid, oligomeric nucleic acid, protein, polysaccharide, sugar, peptide, drug, RNA, DNA, microRNA, or plasmid.

6. The assay kit of claim 1, wherein the hydrophilic coated nanoparticles are produced in a continuous flow chemistry process.

7. The assay kit of claim 1, wherein the hydrophilic coated nanoparticles are non-spherical quantum dots comprising a core material surrounded by a shell material that is produced using a continuous flow chemistry process.

8. The assay kit of claim 1, wherein the hydrophilic coated nanoparticles are non-spherical quantum dots comprising a core material surrounded by a shell material that is aqueous solubilized and produced using a continuous flow chemistry process.

9. The assay kit in claim 1, wherein the hydrophilic coated nanoparticles are non-spherical quantum dots comprising a core material surrounded by a shell material that is aqueous solubilized, paired to an antibody, and produced a using continuous flow chemistry process

10. The assay kit of claim 1, wherein the biological material is at least one of of: CD4 antibody, CD25 antibody, CD17 anitbody, TLR antibody, FoxP3 antibody, or methyl antibody.

11. The assay kit of claim 1, wherein the predetermined target cell types are any one or more of: T regulatory cells, TH17 cells, T-cells or Toll-like receptors present on cells.

12. The assay kit of claim 1, further comprising at least two nanoplex, wherein each nanoplex emits at a different wavelength.

13. The assay kit of claim 1, wherein the nanoplexes are packaged as a Z-plex assay kit, wherein Z is an integer.

14. The assay kit of claim 1, adapted for use in at least one of a lateral flow assay, ELISA, sandwich ELISA, microarray, piezoarray, Western blot, flow cytometry, or UV spectromphotometer.

15. A method of producing an assay kit comprising:
coating a set of nanoparticles to form a set of hydrophilic coated nanoparticles;
pairing the V set of hydrophilic nanoparticles of one or more wavelengths to W biological materials to form X nanoplexes; V, W and X are integers.
